(19) United States
(12) Patent Application Publication
(10) Pub. No.: US 2024/0165038 A1
(43) Pub. Date: May 23, 2024

(54) PULLULAN CAPSULES

(71) Applicant: Capsugel Belgium NV, Bornem (BE)

(72) Inventors: Claire Tardy, Urschenheim (FR); Stefaan Vanquickenborne, Rijmenam (BE)

(73) Assignee: Capsugel Belgium NV, Bornem (BE)

(21) Appl. No.: 18/549,330

(22) PCT Filed: Jan. 12, 2022
(86) PCT No.: PCT/EP2022/050500

§ 371 Date: Sep. 6, 2023

(30) Foreign Application Priority Data
Mar. 12, 2021 (EP) 21162354.1

(51) Int. Cl. A61K 9/48; A61K 47/36

(52) U.S. Cl. CPC A61K 9/4816; A61K 47/36

(57) ABSTRACT

The invention discloses a hard capsule shell comprising pullulan, low acyl gellan, high acyl gellan and KCl.

### BACKGROUND OF THE INVENTION

Hard capsules are used as dosage forms for pharmaceuticals or nutraceuticals.

U.S. Pat. No. 3,258,115 discloses a two-piece hard gelatin capsule, the gelatin forms the shell of the capsule.

Gelatin is commonly derived from collagen taken from animal body parts which is not acceptable for vegetarians.

There as a need for a hard capsule which does not contain gelatin in its shell but instead a polymer from non-animal source.

This need was met by a hard capsule made of pullulan.

### Abbreviations and Definitions Used in this Specification

API active pharmaceutical ingredient
CAP cellulose acetate phthalate
HAG High acyl gellan, CAS 71010-52-1, also called native gellan with two acyl substituents acetate and glycerate
HPMC hydroxypropyl methylcellulose, also called hypromellose or Cellulose, 2-hydroxypropyl methyl ether or cellulose hydroxypropyl methyl ether, CAS 9004-65-3
HPMCAS hydroxypropyl methylcellulose acetate succinate, hypromellose acetate succinate
HPMCP hydroxypropyl methylcellulose phthalate
LAG Low acyl gellan, CAS 71010-52-1, prepared from High acyl gellan by removing the acyl groups.
LOD loss on drying
pullulan CAS 9057-02-7
PVA polyvinyl alcohol
SE Sucrose Ester: Sucrose Monolaurate, CAS 25339-99-5
SML sorbitan mono laurate, CAS 1338-39-2

### SUMMARY OF THE INVENTION

Subject of the invention is a hard capsule shell, CAPSSHELL, comprising
95 to 98.7 wt % of pullulan;
0.2 to 0.5 wt % of low acyl gellan;
0.1 to 0.5 wt % of high acyl gellan;
1 to 2 wt % of KCl;

with the wt % being based on the dry capsule weight.

### DETAILED DESCRIPTION OF THE INVENTION

CAPSSHELL is a two piece hard capsule shell. The two pieces are the cap and the body of the capsule shell which are telescopically joined with each other when closing CAPSSHELL.

Preferably, CAPSSHELL does not comprise gelatin, HPMC, PVA or modified starch.

Preferably, CAPSSHELL does not comprise a polymer selected from the group of HPMCAS, HPMCP, CAP, and polyacrylic acid copolymers.

More preferably, CAPSSHELL does not comprise any other film forming polymer besides pullulan.

Low acyl gellan, high acyl gellan and their combination are known to the skilled person as being gelling agents.

KCl, when used in combination with a gelling agent, is known to the skilled person as being a gelling aid. So the combination of low acyl gellan, high acyl gellan and KCl in CAPSSHELL is a combination of a gelling agent and a gelling aid.

Preferably, CAPSSHELL does not contain a gelling agent other than the combination of low acyl gellan and high acyl gellan; and CAPSSHELL does not contain a combination of a gelling agent with a gelling aid other than the combination of low acyl gellan, high acyl gellan and KCl.

Preferably, CAPSSHELL comprises
95 to 98.4 wt % of pullulan;
0.25 to 0.45 wt % of low acyl gellan;
0.15 to 0.45 wt % of high acyl gellan;
1.2 to 1.8 wt % of KCl;

with the wt % being based on the dry capsule weight.

More preferably, CAPSSHELL comprises
95 to 98.3 wt % of pullulan;
0.3 to 0.4 wt % of low acyl gellan;
0.2 to 0.4 wt % of high acyl gellan;
1.2 to 1.6 wt % of KCl;

with the wt % being based on the dry capsule weight.

In one embodiment, CAPSSHELL further comprises sucrose monolaurate.

Preferably, when CAPSSHELL comprises sucrose monolaurate, then CAPSSHELL comprises from 0.05 to 0.25 wt %, more preferably from 0.1 to 0.2 wt %, of sucrose monolaurate, with the wt % being based on the dry capsule weight.

In one embodiment, CAPSSHELL further comprises sorbitan mono laurate.

Preferably, when CAPSSHELL comprises sorbitan mono laurate, then CAPSSHELL comprises from 0.01 to 0.05 wt %, more preferably from 0.02 to 0.04 wt %, of sorbitan mono laurate, with the wt % being based on the dry capsule weight.

In one embodiment, CAPSSHELL further comprises NaHCO₃.

Preferably, when CAPSSHELL comprises NaHCO₃, then CAPSSHELL comprises from 0.02 to 0.06 wt %, more preferably from 0.03 to 0.05 wt %, of NaHCO₃, with the wt % being based on the dry capsule weight.

CAPSSHELL may comprise water. The water may stem from the production process which may be using an aqueous composition for preparing CAPSSHELL, so the water in CAPSSHELL is typically residual water remaining in CAPSSHELL after drying.

The water may also stem from the humidity of the atmosphere, essentially of the relative humidity of the air, surrounding CAPSSHELL. Typical upper limit of the amount of residual water in CAPSSHELL is 20 wt % or less, preferably 15 wt % or less, more preferably 10 wt % or less, the wt % being based on the weight of the CAPSSHELL.

Typical lower limit of the amount of residual water in CAPSSHELL is 1 wt % or more, preferably 2 wt % or more, more preferably 3 wt % or more, the wt % being based on the weight of the CAPSSHELL.

Any of the lower limit of the amount of residual water may be combined with any of the upper limit of the amount of residual water.

For example, the amount of residual water in CAPSSHELL may be from 1 to 20 wt %, preferably from 2 to 15 wt %, more preferably from 3 to 10 wt %, the wt % being based on the weight of the CAPSSHELL.

The amount of residual water may be characterized by the loss on drying, LOD. The dry capsule weight is the capsule weight minus the weight that is lost when the LOD is determined, that is the remaining capsule weight after the LOD determination.

In one embodiment, CAPSSHELL consists of pullulan, low acyl gellan, high acyl gellan, KCl, sucrose monolaurate, sorbitan mono laurate, NaHCO₃ and water; preferably, CAPSSHELL contains at least 95 wt % of pullulan, with the wt % being based on the weight of the CAPSSHELL.

Further subject of the invention is a method for preparation of CAPSSHEL, wherein CAPSSHELL is prepared by dip molding;

with CAPSSHELL as defined herein, also with all its embodiments.

In dip molding an aqueous mixture of the components of CAPSSHELL, this aqueous mixture is called melt.

A mold pin is dipped into the melt and then extracted from the melt. The melt on the outer surface of the mold pin solidifies to form a film, which is still a wet gel. Then the film is dried at 20 to 80° C. After drying, unnecessary portions thereof are cut off to produce a body and a cap respectively, for producing a body a respectively shaped mold pin is used for the dip molding, and for producing the cap a respectively shaped mold pin is used for the dip molding. A pair of the body and the cap constitutes CAPSSHELL; by telescopically engaging the cap with the body a closed CAPSSHELL is obtained.

Further subject of the invention is CAPSSHELL filled with an active agent, AA, with AA being selected from the group drug, medicament, pharmaceutical, therapeutic agent, nutraceutical, and active pharmaceutical ingredient, API;

with CAPSSHELL as defined herein, also with all its embodiments.

### EXAMPLES

### Definitions, Materials, Abbreviations and Methods Used in this Specification

DM demineralized
HAG High acyl gellan, CAS 71010-52-1, KELCOGEL® CG-HA Gellan Gum, from CPKelco, Atlanta, USA
Indigocarmin CAS-Nummer 860-22-0, Dye content 85%, SigmaAldrich
KCl Potassium Chloride, CAS 7440-09-7, VWR Chemicals
LAG Low acyl gellan, CAS 71010-52-1, KELCOGEL® CG-LA Gellan Gum, from CPKelco, Atlanta, USA
LOD loss on drying
NaHCO₃Sodium bicarbonate, CAS 144-55-8, Sigma Aldrich
PGC Plantcaps Gellan Capsules
PC Standard Plantcaps Capsules, PLANTCAPS® CAPSULES of Capsugel, now a Lonza company, Lonza Ltd, 3930 Visp, Switzerland
Pullulan Pullulan PI-20, CAS 9057-02-7, from Hayashibara Co., Ltd., Japan
SE Sucrose Ester: Sucrose Monolaurate (L-1695), CAS 25339-99-5, Mitsubishi Chemical Corporation, Japan
SML sorbitan mono laurate, CAS 1338-39-2, Lonza Ltd., Visp, Switzerland
USP United States Pharmacopeia

Tube Tester

developed in-house by Capsugel, now Lonza Ltd, Visp, Switzerland:

Method:

The capsule's fracture and elasticity behavior is measured by its resistance to an impact test with a tube.

Procedure:

1. Store the capsules for five days before testing in desiccators.

Storage conditions available are: 10, 23, 33, 45% RH

Store 50 capsules for each condition in opened boxes. Closes it thereafter to avoid any moisture exchange with the surrounding atmosphere.

2. Place a capsule horizontally on a flat surface.

3. Put the weight of 100 g in the tube.

4. Place the tube on the capsule and push the latch to release the weight.

5. Test 50 capsules.

Use closed boxes for storing after equilibration with chosen RH, and do not take out every 50 capsules at the same time (to avoid any humidity retaking).

Record the number of body, cap or capsule (body and cap) broken.

Media Preparation

pH 1.2 USP:
Dissolve 2 g of sodium chloride in 700 ml of demineralized water.
Add 7 ml of concentrated hydrochloric acid (Merck, 37% in water) under stirring.
Complete with demineralized water to 1000 ml.

DM Water USP:
from Elix® 20 Water Purification System, Millipore, Merck KGaA, Darmstadt, Germany

pH 6.8 USP:
Solution A: KH₂PO₄ 0.2 M
Dissolve 27.22 g of potassium dihydrogen phosphate (KH2PO4) in 1000 ml of demineralized water
Solution B: NaOH 0.2 M
Dissolve 8.0 g of sodium hydroxide (NaOH) in 1000 ml of demineralized water
pH 6.8 USP:
Mix 250 ml of solution A and 112 ml of solution B.
Complete with demineralized water to 1000 ml

### Example 1: Preparation of Plantcaps Gellan Melt

TABLE 1

| | | [kg] |
|---|---|---|
| Step 1 | Preparation of gellan melt | |
| | LAG | 0.35 |
| | HAG | 0.3 |
| | NaHCO3 | 0.04 |
| | Ion exchanged water | 335.3 |
| | Total weight | 335.99 |
| Step 2 | Preparation of solution of KCl | |
| | KC1 | 1.5 |
| | water | 13.5 |
| | Total weight | 15 |
| | Content: 10 wt % KCl based on weight of solution | |
| | of KCl | |
| Step 3 | Preparation of solution of surfactants | |
| Step 3a | Preparation of SE solution | |
| | SE | 0.14 |
| | Water | 1.26 |
| | Total | 1.4 |
| | Content: 10 wt % SE based on weight of SE solution | |
| Step 3b | Preparation of SML solution | |
| | SML | 0.03 |
| | Water | 0.27 |
| | Total | 0.3 |
| | Content: 10 wt % SML based on weight of SML | |
| | solution | |
| Step 4 | Preparation of pullulan melt | |
| | gellan melt, prepared according to Step 1 | 335.99 |
| | Solution of KCl, prepared according to Step 2 | 15 |
| | pullulan | 100 |
| | Total weight | 450.99 |

Preparation of gellan melt in Step 1 was done by filling a vessel with ion exchanged water of 80 to 85° C. into which NaHCO3 was charged and mixed for 5 min. A blend of gellans (LAG and HAG) was then added and mixed for 200 min.

A solution of 10% KCl, prepared according to Step 2, was added into the vessel which was then cooled to 68° C..

Then pullulan was charged and the mixture was mixed for 60 min. Step 4 in Table 1 gives a summary.

The mixture was kept stirring at 68° C. for 60 min for debubbling until no bubbles were observed anymore and then for further 90 min for ageing, providing the pullulan melt.

The pullulan melt was transferred to a transfer tank at 65° C..

1.4 kg of SE solution, prepared according to step 3a, and 0.3 kg of SML solution, prepared according to step 3b, were mixed to provide a surfactant solution

The surfactants solution was added to the pullulan melt in the transfer tank; with gentle mixing and maintaining 65° C., providing the Plantcaps Gellan Melt for further use for dip molding.

Table 1 gives the amounts of the components that were used. Table 2 gives the composition of the Plantcaps Gellan Melt and of the final capsule based on dry weight of capsule.

TABLE 2
Plantcaps Gellan Melt

| | | wt % based on | wt % based on |
|---|---|---|---|
| | Melt | weight of melt | dry capsule weight |
| | [kg] | [wt %] | [wt %] |
| Pullulan | 100 | 22.09 | 97.69 |
| LAG | 0.35 | 0.077 | 0.34 |
| HAG | 0.3 | 0.066 | 0.29 |
| KCl | 1.5 | 0.331 | 1.47 |
| NaHCO3 | 0.04 | 0.009 | 0.04 |
| SE | 0.14 | 0.031 | 0.14 |
| SML | 0.03 | 0.007 | 0.03 |
| Ion exchanged water | 350.33 | 77.389 | — |
| 335.3 + 13.5 + 1.26 + | | | |
| 0.27 = 350.33 | | | |
| Total weight | 452.69 | 100.0 | 100.0 |

### Example 2: Preparation of Plantcaps Gellan Capsules, PGC

Plantcaps Gellan Melt, prepared according to example 1, was used to produce shells in form of capsule halves (bodies and caps) with capsule size 2 and standard target weights (capsule weight 61+/− 4 mg) through conventional dip molding by dipping stainless steel mold pins with a temperature of 22° C. into the Plantcaps Gellan Melt with a temperature of 60° C.. A film was formed on the mold pins. After first drying of the film on the mold pins at 26° C. and 44% RH for 15 to 20 min, a second drying at 32° C. and 25 to 30% RH for 30 min was applied and finally a drying at 22° C. and 50% RH was applied before stripping the capsule parts from the metal pins.

The capsule halves were removed from the mold pins.

Capsules were assembled by joining always one cap with one body.

### Example 3: Testing of Capsule—Mechanical Performance

The fracture behavior of the capsules, prepared according to Example 2, was measured by their resistance to an impact test with a tube tester. In preparation of the test the capsules were stored for equilibration for five days under four storage conditions in desiccators: 10, 23, 33 and 45% RH in order to obtain capsules with different LOD. The number of capsules tested was 50 per each RH value and the number of broken bodies or broken caps was recorded. Percentage of broken capsules is presented in Table 3.

TABLE 3
Capsules:
PGC Plantcaps Gellan Capsules prepared according to Example 2

| | Broken Capsules | RH of Storage Condition |
|---|---|---|
| | [%] | [%] |
| PGC | 100 | 2.5 |
| | 58 | 10 |
| | 2 | 16 |
| | 0 | 23 |
| | 0 | 33 |
| | 0 | 45 |

The mechanical performance is similar for the PGC and the PC, the deviation is within the usual variation of the tube test.

### Example 4: Testing of Capsules—Disintegration of Capsules

Capsules, prepared according to Example 2, were tested for disintegration in a disintegration tester, Sotax DT2, SOTAX AG, 4147 Aesch, Switzerland, in accordance to the USP specifications.

For this test the capsules were filled with 320+/−10 mg of a blend of Lactose containing 0.1 wt % of Indigocarmin dye.

The capsules were placed in the different media at 37° C.+/−1° C. and the time of first leak, of capsule emptying and of total disintegration of the capsule were recorded.

6 capsules per media were tested and the respective average time was calculated.

Results are given in Table 4

TABLE 4
Capsules:
PGC Plantcaps Gellan Capsules prepared according to Example 2

| | | Time [min] | | |
|---|---|---|---|---|
| | | pH 1.2 USP | DM water USP | pH 6.8 USP |
| PGC | First leak | 02:51 | 01:53 | 02:05 |
| | Capsule | 08:55 | 04:27 | 05:19 |
| | emptied | | | |
| | Total | 14:34 | 07:17 | 08:28 |
| | disintegration | | | |

### Example 5: Testing on Capsules—Dissolution of Capsules

The capsules, prepared according to Example 2, were tested for dissolution performance in different media: pH 1.2 USP, DM water USP and pH 6.8 USP using a Sotax AT70smart, SOTAX AG, 4147 Aesch, Switzerland, dissolution bath at 37° C. with paddle at 50 rpm, acetaminophen dissolution was followed with Perkin Elmer Lambda 25 UV/VIS spectrometer at 300 nm wavelength. Capsules were filled with Acetaminophen (APAP, Sigma Aldrich Ref A5000).

Table 5 shows the results as measured % of dissolution of the APAP contained in the capsules over time.

TABLE 5
Abbreviations in Table 5:
PGC Plantcaps Gellan Capsules prepared according to Example 2
M Media:
1.2 pH 1.2 USP
DM DM water USP
6.8 pH 6.8 USP

| | | time [min] | | | | | | | | | | | |
|---|---|---|---|---|---|---|---|---|---|---|---|---|---|
| | | 0 | 3 | 6 | 9 | 12 | 15 | 20 | 25 | 30 | 45 | 60 | 75 |
| | M | | | | | | | | | | | | |
| PGC | 1.2 | 0 | 0.2 | 0.4 | 0.7 | 1.1 | 1.7 | 3.6 | 9.3 | 13.9 | 23.9 | 33.4 | 41.2 |
| | DM | 0 | 0.2 | 1.1 | 7.2 | 13.9 | 20.6 | 31.3 | 43.0 | 52.6 | 77.6 | 91.3 | 97.6 |
| | 6.8 | 0 | 0.1 | 0.3 | 0.6 | 0.9 | 1.2 | 1.7 | 4.4 | 6.5 | 22.4 | 32.2 | 43.2 |

1. A hard capsule shell, comprising
95 to 98.7 wt % of pullulan;
0.2 to 0.5 wt % of low acyl gellan;
0.1 to 0.5 wt % of high acyl gellan;
1 to 2 wt % of KCl;
with the wt % being based on the dry capsule weight.

2. The hard capsule shell according to claim 1, wherein the hard capsule shell does not comprise gelatin, hydroxypropyl methylcellulose (HPMC), polyvinyl alcohol (PVA), or modified starch.

3. The hard capsule shell according to claim 1, wherein the hard capsule shell does not comprise a polymer selected from the group of hydroxypropyl methylcellulose acetate succinate (HPMCAS), hydroxypropyl methylcellulose phthalate (HPMCP), cellulose acetate phthalate (CAP), and polyacrylic acid copolymers.

4. The hard capsule shell according to claim 1, wherein the hard capsule shell does not comprise any other film forming polymer besides pullulan.

5. The hard capsule shell according to claim 1, wherein:
the hard capsule shell does not contain a gelling agent other than the combination of low acyl gellan and high acyl gellan; and
the hard capsule shell does not contain a combination of a gelling agent with a gelling aid other than the combination of low acyl gellan, high acyl gellan and KCl.

6. The hard capsule shell according to claim 1, wherein the hard capsule shell comprises:
95 to 98.4 wt % of pullulan;
0.25 to 0.45 wt % of low acyl gellan;
0.15 to 0.45 wt % of high acyl gellan;
1.2 to 1.8 wt % of KCl;
with the wt % being based on the dry capsule weight.

7. The hard capsule shell according to claim 1, wherein the hard capsule shell comprises:
95 to 98.3 wt % of pullulan;
0.3 to 0.4 wt % of low acyl gellan;
0.2 to 0.4 wt % of high acyl gellan;
1.2 to 1.6 wt % of KCl;
with the wt % being based on the dry capsule weight.

8. The hard capsule shell according to claim 1, wherein the hard capsule shell further comprises sucrose monolaurate.

9. The hard capsule shell according to claim 8, wherein the hard capsule shell comprises from 0.05 to 0.25 wt % of sucrose monolaurate, with the wt % being based on the dry capsule weight.

10. The hard capsule shell according to claim 1, wherein the hard capsule shell further comprises sorbitan mono laurate.

11. The hard capsule shell according to claim 10, wherein the hard capsule shell comprises from 0.01 to 0.05 wt % of sorbitan mono laurate, with the wt % being based on the dry capsule weight.

12. The hard capsule shell according claim 1, wherein the hard capsule shell further comprises NaHCO₃.

13. The hard capsule shell according to claim 12, wherein the hard capsule shell comprises from 0.02 to 0.06 wt % of NaHCO₃, with the wt % being based on the dry capsule weight.

14. The hard capsule shell according to claim 1, wherein the hard capsule shell consists of pullulan, low acyl gellan, high acyl gellan, KCl, sucrose monolaurate, sorbitan mono laurate, NaHCO₃ and water.

15. The hard capsule shell according to claim 14, wherein the hard capsule shell contains at least 95 wt % of pullulan, with the wt % being based on the weight of the hard capsule shell.

16. A method for preparation of a hard capsule shell, wherein
preparing the hard capsule shell by dip molding,
with the hard capsule shell as defined in claim 1.

17. A hard capsule shell filled with an active agent, with the active agent being a drug, a medicament, a pharmaceutical, therapeutic agent, a nutraceutical, or an active pharmaceutical ingredient, or any combination thereof,
with the hard capsule shell as defined in claim 1.

18. The hard capsule shell according to claim 2, wherein the hard capsule shell:
(i) does not comprise HPMCAS, HPMCP, CAP, or a polyacrylic acid copolymer; or
(ii) does not comprise any other film forming polymer besides pullulan; or
(iii) both (i) and (ii).

19. The hard capsule shell according to claim 18, wherein:
the hard capsule shell does not contain a gelling agent other than the combination of low acyl gellan and high acyl gellan; and
the hard capsule shell does not contain a combination of a gelling agent with a gelling aid other than the combination of low acyl gellan, high acyl gellan and KCl.

20. The hard capsule shell according to claim 18, wherein the hard capsule shell further comprises sucrose monolaurate, sorbitan mono laurate, or a combination thereof.
